(19) United States
(12) Patent Application Publication
(10) Pub. No.: US 2008/0161915 A1
(43) Pub. Date: Jul. 3, 2008

(54) SYSTEM FOR CREATING VISUAL IMAGES

(76) Inventor: Qiushi Ren, Shanghai (CN)

Correspondence Address: NEIL K. NYDEGGER;NYDEGGER & ASSOCIATES, 348 Olive Street, San Diego, CA, 92103, US

(21) Appl. No.: 11/618,171

(22) Filed: Dec. 29, 2006

(51) Int. Cl. A61F 2/14

(52) U.S. Cl. 623/6.64

(57) ABSTRACT

A system for generating visual images includes an artificial eye that is surgically implanted within a patient's orbit. Also, the system includes a micro-camera that is mounted to the artificial eye for capturing visual input. A digital signal processor receives the visual input from the micro-camera and generates electrical stimuli corresponding to pixels of the visual input. In the system, a plurality of electrode arrays are embedded into selected areas of the primary visual cortex of the patient. Further, each array is interconnected with the processor to transfer the electrical stimuli to predetermined areas of the primary visual cortex for creation of a visual image by the patient corresponding to the visual input.

### FIELD OF THE INVENTION

The present invention pertains generally to systems for creating visual images in blind persons. More particularly, the present invention pertains to systems and methods for stimulating the primary visual cortex to create visual images. The present invention is particularly, but not exclusively, useful as a system that captures visual data with a micro-camera mounted in an artificial eye, processes the data to generate electrical stimuli, and then transfers the electrical stimuli to predetermined areas of the primary visual cortex for the creation of a visual image corresponding to the visual data.

### BACKGROUND OF THE INVENTION

With a normal eye, the visual perception of an object results when light is anatomically converted into impulse signals that will neurologically stimulate the brain. In overview, this conversion essentially involves three separate, but interrelated, functions. First, light from the object that is being viewed, needs to be focused in order to create an image. In an eye, this focusing is done by the eye's lens and its associated anatomy. Secondly, the focused image is converted into neurological impulse signals by the retina (fundus). In this function, the retina actually works together with a complex nerve structure that is associated with the retina. Finally, as the third defined function, the neurological impulse signals are transferred from the retina to a portion of the central nervous system in the brain (primary visual cortex). This transfer is accomplished by the optic nerve. When any part of this anatomical optical system becomes inoperable or dysfunctional, for whatever reason, intervention becomes necessary in order to restore sight. Fortunately, some situations yield to modern medical treatments. Others, however, do not. Of particular interest here are the situations wherein the eye and the optic nerve become essentially ineffective and unusable for their intended purposes. In such situations, the primary visual cortex may be manipulated to create visual images despite the condition of the eye and optic nerve.

Anatomically, the primary visual cortex is the region of the cerebral cortex occupying the entire surface of the occipital lobe. In a person with normal vision, it receives visual data from the retina through the lateral geniculate body of the thalamus. Of importance here is the fact that the primary visual cortex receives bio-electrical stimuli from the lateral geniculate body to create a sensation of light. It is known that these stimuli can be generated by responses to light or by other means. For example, a phosphene, by definition, is a sensation of light that is caused by excitation of the neurological tissues along the visual pathway by mechanical or electrical means, rather than by light.

In light of the above, it is an object of the present invention to provide a system, and a method for its use, that stimulates the primary visual cortex with embedded electrodes in response to stimuli to create visual images corresponding to an object. Another object of the present invention is to provide a system that utilizes an artificial eye with a micro-camera to capture visual data that is electronically, and non-bioelectrically, communicated to the primary visual cortex. Another object of the present invention is to provide a system and method that can effectively bypass a diseased or dysfunctional retina and optic nerve, and still provide a patient with the ability to create a visual image corresponding to an object. Yet another object of the present invention is to provide a visual prosthesis, and a method for its use, that is simple to use, relatively easy to manufacture, and comparatively cost effective.

### SUMMARY OF THE INVENTION

In accordance with the present invention, a system directly stimulates the primary visual cortex of an otherwise blind patient to create a visual image by the patient. Importantly, visual input is captured and converted into electrical stimuli. This electrical stimuli is then transferred to predetermined areas of the primary visual cortex for creation of the visual image by the patient.

In detail, the system includes an implantable artificial eye, preferably comprised of coral, bio-compatible ceramics, or PMMA. Further, the system includes a micro-camera mounted to the artificial eye for capturing visual input or data of the object being viewed. Preferably, the camera is a CMOS-technology-based camera, and it is situated in the posterior of the artificial eye. A wide-angle auto-focus lens may be mounted on the micro-camera to focus light from the object onto the camera.

For one embodiment of the present invention, a disk-like solar panel is mounted to the artificial eye to provide power to the micro-camera and its functional circuitry. In other embodiments of the present invention, the micro-camera can be battery powered or RF powered (wireless). As intended for the system, the visual input that is captured by the camera comprises a discreet number of pixels, wherein each pixel is characterized by an intensity and a predetermined position that is representative of the object being viewed.

In the system, a digital signal processor is positioned to electronically receive the visual input from the micro-camera. As envisioned for the present system, the processor may be mounted to the artificial eye or it may be extracorporeal. In either case, the processor is powered by the power supply to process the visual input and generate electrical stimuli corresponding to the pixels of the visual input. Specifically, the electrical stimuli include information that characterizes the intensity and position of the pixels.

Further, the system includes a plurality of electrode arrays. Structurally, the electrodes of each array are embedded directly into selected areas of the primary visual cortex of the patient. Preferably, a plurality of electrode arrays are located on the left cortex and a plurality of electrode arrays are located on the right cortex of the patient. Also, each array is electrically interconnected with the processor for transfer of the electrical stimuli to predetermined areas of the primary visual cortex for creation of the visual image. If the processor is extracorporeal, the electrical stimuli may be modulated onto an electromagnetic radio frequency carrier. Otherwise, the processor is connected to the electrode arrays by a cable.

Structurally, each electrode array includes a base member having a plurality of probes that are mounted on the base member for embedment into the primary visual cortex. At least one electrode is mounted on each probe, and each electrode on the probe is in electrical communication with the processor. Specifically, each electrode receives a portion of the electrical stimuli.

In greater structural detail, the electrode array for the present invention may include a plurality of probes that are aligned in a plurality of rows on the base member. Further, there may be a plurality of base members in the array. Regardless how many base members and how many probes there may be, each probe will preferably have a length that is in a range between five hundred micrometers and ten millimeters (500 μm-10 mm). Also, the probes can be spaced from adjacent probes by a distance that is in a range between ten micrometers and five millimeters (10 μm-5 mm).

Further, each probe may be a multi-filament bundled electrode. This type of electrode includes a center pillar probe that is surrounded by a plurality of single filament electrodes. Further, the center pillar probe preferably has a diameter and a length greater than the single filament electrodes have. The greater diameter and length provides the center pillar probe with the mechanical strength needed to penetrate neural tissues and to facilitate engagement between the multi-filament electrode and the primary visual cortex.

In order to provide a blind patient with the ability to create visual images, an artificial eye including a micro-camera is surgically implanted into a patient's orbit. Further, a plurality of electrode arrays are embedded into selected areas of the primary visual cortex of the patient. Also, a signal processor is electronically interconnected between the micro-camera and the electrode arrays.

During operation of the present system, visual data is initially captured by the micro-camera. Thereafter, the visual data is communicated to the processor which then generates corresponding electrical stimuli. Specifically, generation of the electrical stimuli corresponding to the pixels of the visual input is accomplished in accordance with a predetermined data protocol. As recognized and appreciated by the present system, this data protocol is patient specific and will differ from one patient to another. Consequently, the predetermined data protocol that is used to generate the electrical stimuli is customized for the particular patient. This is done in post-operative examination, testing and training sessions where the data protocol is established so that each pixel address from the visual input will correspond with a specific location in the primary visual cortex.

After the processor has generated the electrical stimuli, the stimuli is transferred through the electrode arrays to the predetermined areas of the primary visual cortex. The primary visual cortex then creates a visual image of the object being viewed.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of the anatomical components involved in visual perception;

FIG. 2 is a perspective view of the system for creating visual images in accordance with the present invention;

FIG. 3 is a rear view of the electrode arrays of FIG. 2 implanted on the primary visual cortex;

FIG. 4A is a side view of an electrode array for use in the system of the present invention;

FIG. 4B is a cross-sectional view of the electrode array as would be seen along the line 4B-4B in FIG. 4A;

FIG. 5A is a perspective view of an embodiment of an electrode for use in the electrode array of the present invention;

FIG. 5B is a perspective view of an electrode array having electrodes as illustrated in FIG. 5A; and

FIG. 5C is a cross-sectional view of the electrode array as would be seen along the line 5C-5C in FIG. 5B.

### DESCRIPTION OF THE PREFERRED EMBODIMENTS

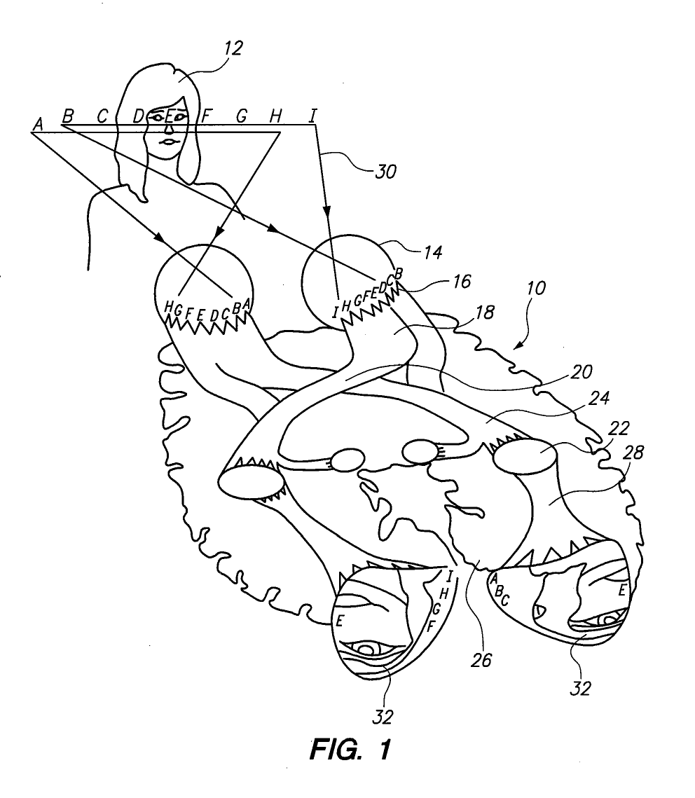

Referring initially to FIG. 1, the anatomical components, generally designated 10, providing for visual perception of an object 12 are illustrated. As shown, visual perception begins at the eyes 14 which focus light on the retinas 16. At the posterior of the eyes 14, optic nerves 18 are connected to the retinas 16. As shown, the optic nerves 18 cross one another at the optic chiasma 20. After crossing one another, the optic nerves 18 are connected to the lateral geniculate nuclei 22 by optic tracts 24. Further, the lateral geniculate nuclei 22 are connected to the primary visual cortex 26 by optic radiations 28.

During visual perception of the object 12, light (represented by arrows 30) enters each eye 14 and is focused on each retina 16. As shown by letters A-I, different areas of the object 12 are received by different physical positions in the retinas 16. After the light 30 is converted bio-electrically to electrical pulses and communicated to the primary visual cortex 26 through the other anatomical components 10, a visual perception of the object 12, or visual image 32 is created at the primary visual cortex 26. As shown, the visual image 32 is vertically and horizontally inverted compared to orientation of the actual object 12.

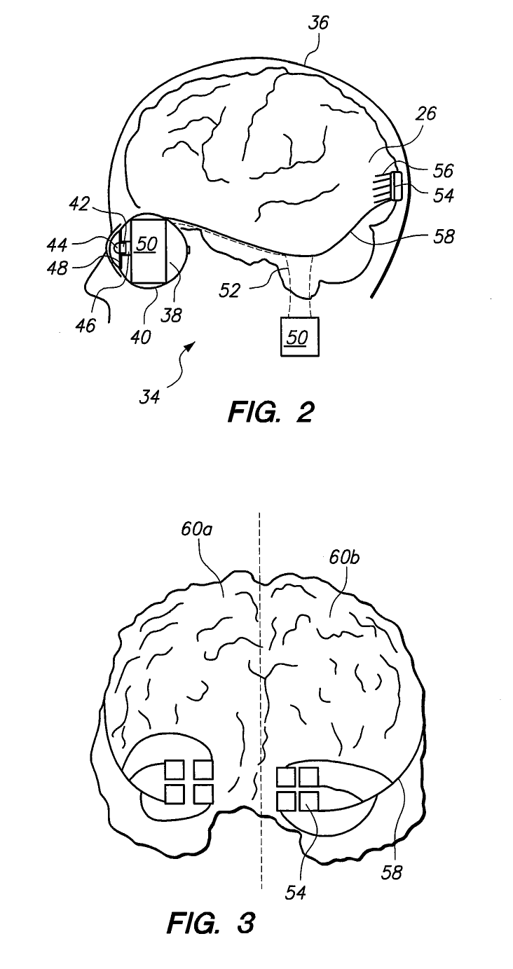

With this understanding of normal anatomical performance, FIG. 2 can be more clearly understood. In FIG. 2, a system for creating a visual image 32 in accordance with the present invention is shown and is generally designated 34. With the system 34 shown, an otherwise blind patient 36 is provided with the ability to create visual images 32 in his primary visual cortex 26. To provide this capability, the system 34 includes an artificial eye 38 for capturing visual data, e.g., light. Preferably, the artificial eye 38 is made from coral, bio-compatible ceramics or PMMA. As shown, the artificial eye is held in the patient's orbit 40.

Still referring to FIG. 2, it can be seen that the system 34 includes an intraocular (CMOS) camera 42 that is mounted on the artificial eye 38. Further, the camera 42 includes a wide angle lens 44. Preferably, the camera 42 is a CMOS camera having a sensor area 3.37 mm×2.54 mm, with pixel sizes of 2.0 μm×2.0 μm and an output of 25 frames per second at 128×128 pixel resolution. Other similar type cameras with different pixel size and resolution, however, could be used as well. As envisioned for the present invention, the various types of CMOS cameras that can be used as the camera 42 will vary primarily in the type of power supply that is used. Referring still to FIG. 2 it will be seen that the camera 42 typically includes a power pack 46 that provides operational power for the camera 42. With this combination, at least three different sources of power are envisioned for camera 42. For one, power can be supplied to the camera 42 via a transcutaneous connection. In particular, such a connection will likely be with an extracorporeal voltage source, such as a battery (not shown). For another, the camera 42 may include an energy transfer unit 48 that is effectively mounted on the artificial eye 38. In one embodiment, this energy transfer unit 48 can be a solar panel that will transfer energy to the power pack 46 for subsequent use. In another embodiment, the energy transfer unit 48 may be an RF antenna that will pass energy via a wireless connection to the power pack 46. In any event, the present invention envisions several different type devices for powering the camera 42.

As further shown in FIG. 2, the system 34 includes a digital signal processor 50 that is connected to the camera 42. For purposes of the present invention, the signal processor 50 includes an opto-electrical transfer mechanism for converting visual data to an electronic format. As shown in FIG. 2, the processor 50 may be mounted in the artificial eye 38, mounted elsewhere in the patient's body, or it may be extracorporeal (shown in phantom). For a signal processor 50 mounted outside of the artificial eye 38, the system 34 includes a percutaneous or transcutaneous electronic line 52 that connects the intraocular camera 42 with the signal processor 50. For an extracorporeal processor 50, the processor 50 and camera 42 may include antennas (not shown) that communicate through a modulated, radio frequency, transmit signal.

Still referring to FIG. 2, it can be seen that the system 34 includes electrode arrays 54 that are intracranially implanted at the patient's primary visual cortex 26. Specifically, each electrode array 54 includes electrodes 56 that penetrate the primary visual cortex 26 to embed the electrode array 54 therein. As further shown, for embodiments including processors 50 that are mounted within the artificial eye 38 or implanted elsewhere in the patient 36, the system 34 includes a percutaneous wire 58 that interconnects the processor 50 and the electrode arrays 54. For an extracorporeal processor 50, the processor 50 and electrode arrays 54 may include antennas (not shown) that communicate through a modulated, radio frequency, transmit signal.

Referring now to FIG. 3, a preferred embodiment of the system 34 is illustrated. As shown, the system 34 includes four electrode arrays 54 for each cortex 60a, b. Specifically, four electrode arrays 54 are embedded in the left cortex 60a and four electrode arrays 54 are embedded in the right cortex 60b . Further, each electrode array 54 is connected to the processor 50 via a respective percutaneous wire 58.

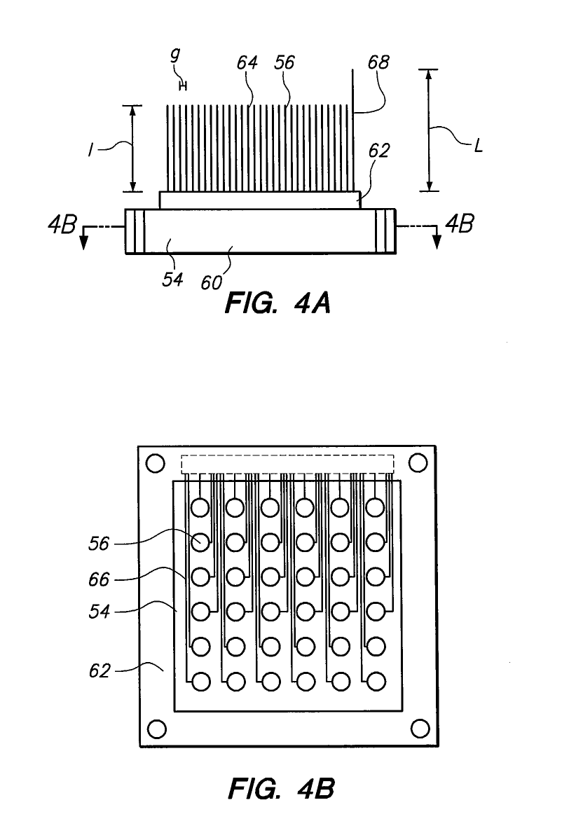

Referring now to FIGS. 4A and 4B, the structure of each electrode array 54 may be understood. As shown in FIG. 4A, the electrode array 54 includes a base member 62. Further, at least one probe 64 is mounted to the base member 62. Preferably, however, there will be a plethora of substantially parallel probes 64 mounted on each base member 62. Also, it is envisioned that the probes 64 will be aligned in columns and rows on the base member 62 (as indicated in FIG. 4B) and that each probe 64 will have a length “I” that is in a range between five hundred micrometers and ten millimeters (500 μm-10 mm). Also, it is envisioned that each probe 64 will be spaced from an adjacent probe 64 by a gap “g” that is in a range between ten micrometers and five millimeters (10 μm-5 mm). Further, each probe 64 will include at least one electrode 56. Most likely, however, there will be a plethora of electrodes 56. Importantly, each electrode 56 will be electronically connected with a respective wire lead 66, and each lead 66 will become part of the percutaneous wire 58 that electronically interconnects the electrode array 54 with the processor 50. As best appreciated with reference to FIG. 2, the probes 64 of electrode array 54 are embedded into the primary visual cortex 26 to establish electrical contacts between the electrodes 56 and the neurons in the primary visual cortex 26. In order to facilitate embedding of the electrode array 54, the array 54 is provided with at least one anchoring probe 68 (see FIG. 4A) that has a length “L” that is greater than the length “I” of the probes 64. Specifically, length “L” is in a range between five hundred micrometers and twelve millimeters (500 μm-12 mm).

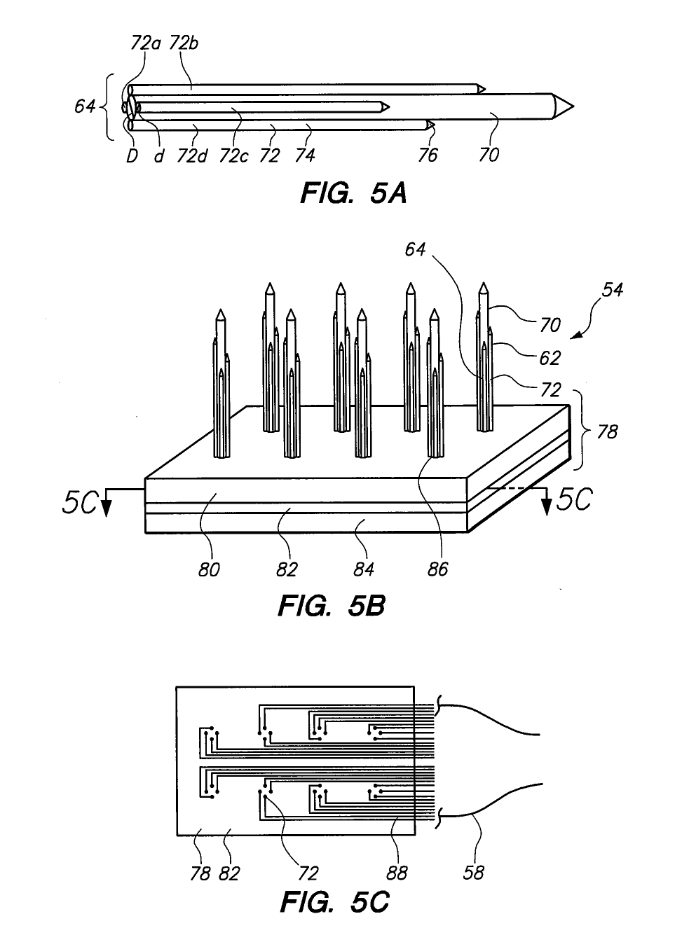

Alternatively, each probe 64 may be a multi-filament bundle that includes a central pillar 70 surrounded by single filament electrodes 72 of varying lengths, as shown in FIG. 5A. For purposes of the present invention, the multi-filament bundle probes 62 are intended to increase efficiency, resolution, and information processing power of the electrode arrays 54. Structurally, the pillar 70 and electrodes 72 of the multi-filament bundle probes 64 are connected by bonding with a medical grade epoxy, by braising, or by micro-fabrication technology. Regardless of the method of connection, the single filament electrodes 72a-d have diameters “d” in a range between ten and two-hundred micrometers (10-200 μm). Further, the central pillar 70 has a diameter “D” in a range between twenty and one-thousand micrometers (20-1000 μm), or roughly two to ten times greater than the diameter “d”. With this structure, the central pillar 70 provides the mechanical strength needed to penetrate the neural tissues at the primary visual cortex 26 and to facilitate penetration of the single filament electrodes 72 into the neural tissue.

Preferably, each single filament electrode 72 is made of a material that is well established for use with neural tissue. Such materials include platinum, iridium, platinum/iridium alloy, gold, and the like. Further, each electrode 72 is insulated with materials 74 such as parylene-C, polyimide, Teflon, and the like to expose only the tip 76 of the electrode 72. In this manner, only the tip 76 of the electrode 72 will contact and provide the electrical stimuli to adjacent neural tissue. Further, the length of the exposed tip 76 is adjustable to match the impedance requirement for stimulation of the adjacent neurons. With this structure, the multi-filament bundle probes 64 provide for increased contact points between the probes 64 and the neural tissue of the primary visual cortex 26, thereby increasing spatial resolution of the visual image 32. Further, due to the varying depths at which the tips 76 of the electrodes 72 are embedded into the primary visual cortex 26, different levels of visual information such as stereo, color, relative movement, and the like, may be evoked by electrical stimuli from the electrodes 72.

Referring now to FIG. 5B, for each array 54, a plurality of probes 64, each comprising a pillar 70 and single filament electrodes 72, are mounted to a base member 78 comprised of bio-compatible ceramics or bio-compatible PMMA plastics. As shown, the base member 78 comprises a mounting plate 80, an intermediate plate 82 and a cover plate 84. Structurally, the mounting plate 80 includes openings 86 through which the probes 64 pass. Referring to FIG. 5C, it can be seen that the single filament electrodes 72 of the probes 64 are electronically connected with respective wire leads 88 that form part of the percutaneous wire 58 that electronically interconnects the electrode array 54 with the processor 50. Specifically, the single filament electrodes 72 and leads 88 are connected at the intermediate plate 82.

For the operation of the system 34, the intraocular camera 42 is aimed by the patient 36. As will be appreciated by the skilled artisan, the camera 42 captures visual data such as light 30 reflected from the object 12, and effectively digitizes the light 30 to create a plurality of discrete pixels. More specifically, each pixel has a location and a light intensity that is characteristic of the object 12 that is being imaged. In a manner well known in the pertinent art, each pixel is converted into an electrical impulse. A plurality of the electrical impulses is then used by the signal processor 50 to create electrical stimuli in accordance with a predetermined data protocol. The predetermined data protocol is essentially an electrical diagram that uses the electrical stimuli to stimulate neurons in the primary visual cortex 26 with an appropriate electrode 56. The consequence of this is the creation of a visual image 32 of the object 12. Anatomically, however, there is no direct correlation between a pixel from the object 12 that is electronically created for the electric stimuli, and a neuron that needs to be stimulated by an electrode 56 for creation of a visual image 32 of an object 12. Such a direct connection would be convenient, but not realistic. Instead, all patients are different. Consequently, an alignment of pixels, such as pixels from an object 12, will inevitably encounter an unpredictable arrangement of neurons. Thus, the electrodes 56 need to be electronically rearranged in order to properly generate visual images 32 in the primary visual cortex 26. For the present invention, this rearrangement of electrode stimulation is done in accordance with the data protocol. And, the data protocol is established by post-operative examination, testing, and training of the patient until the electrical stimuli reliably and properly stimulate neurons in the primary visual cortex 26. Importantly, these stimulations need to faithfully create a visual image 32 of the object 12 for the patient.

While the particular System for Creating Visual Images as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for generating visual images which comprises:
an artificial eye;
a micro-camera mounted to the artificial eye for capturing a visual input;
a digital signal processor for receiving the visual input from the micro-camera and generating electrical stimuli corresponding to pixels of the visual input; and
a plurality of electrode arrays wherein electrodes of each array are embedded into selected areas of the primary visual cortex of a patient, and wherein each array is interconnected with the processor for transfer of the electrical stimuli to predetermined areas of the primary visual cortex for creation of a visual image by the patient corresponding to the visual input.

2. A system as recited in claim 1 wherein the digital signal processor is mounted to the artificial eye.

3. A system as recited in claim 1 further comprising a power supply connected to the digital signal processor.

4. A system as recited in claim 3 wherein the power supply is solar powered.

5. A system as recited in claim 4 wherein the power supply is mounted to the artificial eye.

6. A system as recited in claim 1 wherein the artificial eye is made of a material selected from the group consisting of coral, bio-compatible ceramics, and PMMA.

7. A system as recited in claim 1 wherein a plurality of electrode arrays are located on the left cortex and a plurality of electrode arrays are located on the right cortex of the patient.

8. A system as recited in claim 1 wherein each electrode array includes at least one probe for anchoring the array to the selected area of the primary visual cortex of the patient.

9. A system as recited in claim 1 wherein each electrode includes a pillar electrode and a plurality of single filament electrodes.

10. A system as recited in claim 9 wherein the pillar electrode has a pillar diameter and each of the single filament electrodes have a filament diameter, and wherein the pillar diameter is 2 to 10 times greater than the filament diameter.

11. A system as recited in claim 9 wherein each electrode includes a plurality of single filament electrodes having different lengths.

12. A system for generating visual images which comprises:
an artificial eye;
a means for capturing a visual input, with said capturing means being mounted to the artificial eye;
a means for receiving the visual input from the capturing means and generating electrical stimuli corresponding to pixels of the visual input;
a plurality of electrode arrays, wherein electrodes of each array are embedded into selected areas of the primary visual cortex of a patient; and
a means for interconnecting each array with the receiving means for transfer of the electrical stimuli to predetermined areas of the primary visual cortex for creation of a visual image by the patient corresponding to the visual input.

13. A system as recited in claim 12 wherein the receiving means is mounted to the artificial eye.

14. A system as recited in claim 12 wherein the artificial eye is made of a material selected from the group consisting of coral, bio-compatible ceramics, and PMMA.

15. A system as recited in claim 12 wherein a plurality of electrode arrays are located on the left cortex and a plurality of electrode arrays are located on the right cortex of the patient.

16. A system as recited in claim 12 wherein each electrode includes a pillar electrode and a plurality of single filament electrodes having different lengths.

17. A method for generating visual images which comprises:
surgically implanting an artificial eye to a patient, said artificial eye including a micro-camera;
capturing a visual input with the micro-camera;
communicating the visual input to a signal processor;
generating electrical stimuli corresponding to pixels of the visual input;
embedding a plurality of electrode arrays into selected areas of the primary visual cortex of the patient; and
transferring the electrical stimuli through the electrode arrays to predetermined areas of the primary visual cortex for creation of a visual image by the patient corresponding to the visual input.

18. A method as recited in claim 17 wherein the signal processor is mounted in the artificial eye.

19. A method as recited in claim 17 further comprising the step of connecting a power supply to the signal processor.

20. A method as recited in claim 17 wherein the embedding step includes embedding a plurality of electrode arrays on the left cortex and a plurality of electrode arrays on the right cortex of the patient.
